(19) United States
(12) Patent Application Publication
(10) Pub. No.: US 2003/0082136 A1
(43) Pub. Date: May 1, 2003

(54) Anti-tumor vaccine

(76) Inventors: Bill Minshall, Irvine, CA (US); Suzanne Minshall, Irvine, CA (US); Michael Skotzko, Thousand Oaks, CA (US); John Conner, Sherman Oaks, CA (US)

Correspondence Address: STETINA BRUNDA GARRED & BRUCKER, 75 ENTERPRISE, SUITE 250, ALISO VIEJO, CA, 92656, US

(21) Appl. No.: 10/016,528

(22) Filed: Oct. 30, 2001

(51) Int. Cl. A61K 38/20; A61K 31/715; A61K 39/00

(52) U.S. Cl. 424/85.2; 424/184.1; 424/277.1; 514/54

(57) ABSTRACT

A therapeutic vaccine for the treatment of melanoma, mast cell tumors and sarcoma in mammals. The vaccine preferably comprises at least one allogenic cell line providing at least one, and preferably two or more gangliosides selected from the group consisting of GD-2, GD-3, GM-2, GM-3, GD-1b and GT-1b. At least one cytokine selected from the group consisting of GM-CSF, IL-2, IL-4, and IL-12 and at least one heat shock protein selected from the group consisting of HSP-60, HSP-70 and HSP-90 are added as adjuvants. A bacterial organism may further be added as an adjuvant.

### CROSS-REFERENCE TO RELATED APPLICATIONS

(Not Applicable)

### STATEMENT RE: FEDERALLY SPONSORED RESEARCH/DEVELOPMENT

(Not Applicable)

### BACKGROUND OF THE INVENTION

The present invention is directed to therapeutic vaccines and methods for treating cancerous solid tumors, namely melanoma, carcinoma and sarcoma. The present invention is believed to be particularly well suited for canines, but is further believed to be equally useful in treating such cancers found in felines, equines and possibly human beings.

As is well-known, solid tumors are characterized by a localized mass of tissue comprised of cells which multiply and grow uncontrollably, and ultimately crowd out normal cells. Such tumors can develop in any tissue of any organ and at any age in virtually all species of mammals.

Although all solid tumors possess the ability to invade local tissues and metastasize, the same can be distinguished on the basis of the type of cells the tumors are composed. In this regard, melanomas arise from the skin, mucous membranes, eyes, and central nervous system, where pigment cells occur. Sarcomas arise from connective or supporting tissues, such as bone or muscle. Carcinomas arise from glandular and epithelial cells, which line body tissues.

With respect to the former, melanoma is a cancer associated with the “melanocytes” of the skin, which are the pigment-producing cells that make skin dark. The cancer can appear anywhere on the skin surface, and normally appears in different locations. In human beings, the incidence of malignant melanoma is rising at an alarming rate. In America alone, over 51,000 new cases are expected annually, and the incidence is increasing at a rate of more than seven and one-half percent, higher than any other cancer. Melanoma also has a high incidence in other species of mammals, including canines, which presently comprises fourteen percent of all cancers found in dogs.

Currently, the formation of melanoma tumors are believed to be due to the over-expression of proteins called melanoma-associated antigens (MAAs). Among the melanoma-associated antigens include MAGE-1, MART-1 and GP-100. Other melanoma-specific peptides have been identified, including BAGE, GAGE and tyrosinase. Evidence further suggests additional biological causes of melanoma could include a switch between two types of melanin, which may control the formation of melanomas via a process called “melanogenesis” and further, that two genes of the vitronectin and receptor, a cell surface glycoprotein that promotes the spread of cells and is found at the site of skin lesions and repair, are involved in the development of melanoma.

The progression of melanoma is characterized into four stages. In the first stage, stage I, early in the progression of the disease, melanoma appears as localized primary tumors with a thickness of less than 4 mm. The second stage of melanoma is characterized by localized primary tumors having a thickness greater than 4 mm, with no palpable point of origin. Stage III is characterized by primary tumors of any thickness in regional recurrence. Lastly, in stage IV, melanoma is characterized by primary tumors of any thickness having advanced nodal involvement, or distant metastases (i.e., uncontrolled growth). With respect to this latter stage, prognosis in canines is guarded with most animals showing an overall median survival varying between two and six months. Long-term survival is extremely rare.

As to the treatment of melanoma, surgery is a frequently utilized treatment option for melanoma. Usually, the primary tumor is removed during surgery, followed by a second surgery to ensure removal of all other tumor cells in the area. Interferon, a cytokine which is used to delay or reduce the occurrence in patients with localized melanoma following surgery. The use of Levamisol is also used based upon its ability to produce some immune-enhancement effects. Hyperthermic profusion therapy, which can only be used in patients with a melanoma localized to the limbs, is frequently deployed for individuals with stage II or stage III melanoma.

In addition to the foregoing treatments, recent developments have been made to develop immunotherapy against cancer, including the development of therapeutic cancer vaccines for the treatment of melanoma. Among the approaches currently being developed as a means of vaccinating against melanoma include vaccines aimed at enhancing the antibody response against gangliosides, and in particular, gangliosides GM-2, GD-2 and GD-3. Exemplary of such attempts include those vaccines disclosed in U.S. Pat. No. 5,882,654 issued to Morton on Mar. 16, 1999 entitled Polyvalent Melanoma Vaccine, the teachings of which are expressly incorporated herein by reference.

In this regard, such gangliosides (i.e., glycolipids that contain sialic acid), are over-expressed in melanoma tumors. The gangliosides are introduced into the body via the vaccine in order to elicit an immune response against melanoma-associated antigens. Such approach, however, appears less than promising insofar as such vaccine appeared only to eliminate those tumor cells expressing the injected antigenic ganglioside. As a result, tumor cells that do not express the injected ganglioside or, alternatively, express the same in very low levels, are not targeted and killed via an autoimmune response.

Vaccines incorporating recombinant antigen protein are further being developed. In such approach, melanoma-associated proteins, such as MAGE-1 and GP-100, are used to vaccinate, and thus elicit an immune response thereto. The therapeutic effectiveness of such vaccines at present appears to be uncertain.

A further vaccination approach includes the development of anti-idiotypic antibody vaccines, which attempts to stimulate or suppress the regulation of the host's immune system by mimicking melanoma associated antigens. Essentially, such anti-idiotypic molecules attempt to recognize antibodies specific thereto, in a manner that is the reverse of the recognition of the antigen by the antibody. Such vaccines, however, are based upon an unproven hypothesis, and may generate unnecessary immune responses.

Yet another area of melanoma vaccination involves polyvalent shed antigen vaccine which utilizes cell surface molecules released from melanoma cells to elicit an immune response thereto. Such vaccine is based upon the fact that melanoma cells release approximately half the material on the surface of their cells every three hours. By utilizing such polyvalent shed antigens, initial studies seem to indicate that a clear connection is established between anti-melanoma responses and active cytotoxic responses against melanoma antigens. Concerns arise, however, that the shed antigens do not accurately reflect the true profile of melanoma antigens, and thus create concern over whether or not such vaccine will be effective.

Still further attempts in developing a vaccine for melanoma include cell lysate vaccines that attempt to utilize the components of tumor cells. In this respect, the melanoma cells are treated with a virus which subsequently produces a lysate, or components of the melanoma tumor, that ultimately produce an immune response that, at least in theory, could produce a better, more tumor-specific immune response than antigens administered alone. Although clinical testing is underway, such vaccines may have limited effectiveness and are still under investigation.

Related to melanoma are soft tissue sarcomas, which generally arise from mesenchymal connective tissue. Presently, the etiology of most soft tissue sarcomas remains unknown. With respect to humans, the incidence is approximately 2:100,000 adults, with approximately 5,600 new cases and 1,600 deaths being reported per year in the U.S., and exacting a mortality of 1,600 deaths per year in this country. Sadly, soft tissue sarcomas comprise approximately 6.5% of all tumors found in children, and rank as the fifth cause of death in children under age fifteen. Soft tissue sarcomas also comprise approximately 15% of all canine “skin” and subcutaneous cancers with an annual incidence of approximately 35:100,000 in animals, and include tumor types such as fibrosarcomas and nerve sheath tumors.

In the case of felines, soft tissue sarcomas comprise seven percent of such cancers, with an annual incidence of approximately 17:100,000 animals. Moreover, sarcomas have been increasing in incidence in vaccinated cats, and apparently in places where such vaccines are commonly injected. Indeed, in at least one study, an incidence of approximately 3.6 cases of vaccine site specific sarcoma per 10,000 cats has been estimated in a report cited in the Journal of American Veterinary Medical Association.

As to treatment, soft tissue sarcomas unfortunately have a generally poor response to chemotherapy and radiation therapy. Additionally, soft tissue sarcomas frequently reoccur even after conservative surgical excision, and tend to metastasize in up to 25% of cases.

Although rarely found in humans, mastocytoma (mast cell tumor) comprises another skin neoplasm that is highly prevalent in mammals. Such tumors are among the most common tumors found in dogs and comprise 11-27% of all malignant skin tumors found in dogs, with the boxer, Boston terrier and bulldog breeds having the highest incidences. Mast cell tumors, which is more accurately characterized as a particular type of sarcoma, are comprised of cancerous mast cells. In this regard, mast cells are connective tissue cells that secrete heparin and histamine that are involved in inflammatory responses. Such tumors, which are more accurately characterized as particular types of sarcoma, are comprised of cancerous mast cells. Such tumors are typically found in the skin. Such tumors are also commonly found at visceral sites, including organs such as the spleen, liver and kidneys, which typically indicates a metastatic state of the disease. The larynx, mediastinum and gastrointestinal tract are also primary sites for such tumors.

Mast cell tumors, as per other solid tumors, are characterized in terms of stages. In the first stage, stage I, the mast cell tumor is limited to the dermis, with no lymph node involvement. In the second stage, stage II, the mast cell tumors are confined to the dermis with regional lymph node involvement. Stage III mast cell tumors are characterized by multiple dermal tumors or a large infiltrating tumor with or without lymph node involvement. The final stage, stage IV, is characterized by any tumor with distant metastasis or recurrence with metastasis.

As to the treatment of mast cell tumors, the same involves conventional measures such as surgery, radiation therapy and chemotherapy. With respect to the former, surgery has been shown to be exceptionally effective in treating mast cell tumors diagnosed at an early stage. On the other hand, surgery is substantially ineffective for treating late stage mast cell tumors. Radiation therapy is typically deployed in combination with surgery, and can substantially enhance survival rates to the extent the same is administered in treating early stage mast cell tumors.

As to chemotherapy, regimens have been developed for both the oral and intravenous administration thereof. Intra-lesional therapies have further been developed. Among the most dominant chemotherapeutic agents utilized for treating canine mast cell tumors include glucocorticoids, which may be administered alone or in combination with chlorambucil or, alternatively, in the combination of vinblastine and cyclophosphamides. L-asparaginase is also typically utilized as a chemotherapeutic agent. Triamcinolone is typically deployed for intra-lesional chemotherapy.

Such therapies, however, are well-known to be difficult to administer, can produce serious side effects in the animal, and are typically cost-prohibitive. Even given such treatment modalities, mast cell tumors, if not diagnosed early enough, are practically incurable. In this respect, there has not heretofore been available any sort of treatment, vaccine or otherwise that is available or will potentially become available.

With regard to carcinomas, the same originate from glandular tissue (adenocarcinoma) or the lining of organs. With respect to the latter, carcinomas may take any of a variety of specific forms, including bronchogenic carcinoma, originating in the lungs or airways, cervical carcinoma, originating from the cervix and endometrial carcinoma, originating from the lining of the uterus; numerous other carcinomas are well-known. Carcinomas can additionally originate in the skin, and include basal cell carcinomas—the most common type of skin cancer.

The prevalence of carcinomas varies amongst populations and species. Suffice to say, as per the other cancers discussed above, the same pose a serious health risk and, to the extent the same develop into the latter stages, have little chance of being cured.

Given the prevalence of the aforementioned cancers, as well as the limited effectiveness, cost and side effects associated with currently available treatment regimens, particularly when utilized to treat late-stage cancers, there is a substantial need in the art for treatment, and in particular a therapeutic vaccine that can effectively treat such cancers in mammals, and in particular, canines. There is further a need in the art for such a therapeutic vaccine that invokes a desired immune response that selectively targets the host's tumor cells associated with either melanoma, mast cell tumors and/or sarcoma, utilizing an antigen profile that most ideally stimulates such autoimmune response. There is still further a need in the art for such a therapeutic vaccine that is safe and effective, has the potential to elicit a desired anti-tumor autoimmunne response for a variety of mammalian species, including but not limited to canines, felines, equines, and possibly human beings, and that can further be utilized for the effective treatment of even late stage cancers.

### BRIEF SUMMARY OF THE INVENTION

The present invention specifically addresses and alleviates the above-identified deficiencies in the art. In this respect, the present invention is directed a polyvalent vaccine and method of therapeutically treating cancerous solid tumors, and in particular malignant melanoma, carcinoma, mast cell tumors and sarcomas in mammals. Dogs, felines, equines and human beings are believed to be particularly well-suited recipients of the vaccine and methods of the present invention, although other mammalian species are likely to benefit as well.

According to a preferred embodiment, the vaccine comprises a cell vaccine derived from at least one allogenic cell line which contains a particular profile of tumor-associated antigens in combination with certain adjuvants that promote and augment the host's immune responses. More specifically, the cell line exhibits tumor-associated gangliosides, along with stress proteins, and cytokines in an amount effective to stimulate an immune response collectively thereto that, when taken in combination with certain cytokine and heat shock protein adjuvants, promotes a cytotoxic or cytostatic effect upon the tumor when the vaccine is administered to the host. Such cell line will provide at least one, but preferably two or more tumor-associated gangliosides selected from the group consisting of GD-2, GD-3, GM-2, GM-3, GD-1b, and GT-1b. In a preferred embodiment, such cells may further have Leukocyte Antigens (LA) that are common with the subject to which the vaccine is administered. For canine vaccination, the cellular component may include cell lines such as those produced by Animal Science Laboratories of Palm Desert, Calif. Such cells are rendered incapable of proliferation by irradiation via known techniques.

The vaccine further includes a cytokine adjuvant that promotes immunostimulation which is selected from the group consisting of GM-CSF, IL-2, IL-4, and IL-12, as well as one or more heat shock or stress proteins. Among those well suited include heat shock proteins selected from the group consisting of HSP-60, HSP-70 and HSP-90, with the HSP-90 being preferred. The vaccine may further include a bacterial agent to facilitate the induction of specific immunity to tumors of the aforementioned variety, and may include agents such as Bacillis Calmette-Guerin (BCG).

Although preferably administered as whole-cells, it is contemplated that the aforementioned antigens may be administered via a cell lysate, in combination with the heat shock proteins and cytokines as adjuvants. It is further contemplated that such combination of antigens may be adsorbed upon a suitable pharmaceutically-acceptable carrier well known in the art.

Also embodied in this invention are methods for treating cancerous solid tumors of the aforementioned variety, which comprise administering any one of the vaccines of the present invention, or eliciting an anti-tumor immunological response. According to one preferred method, such vaccine is preferably administered weekly for five weeks, and thereafter monthly for approximately ten to eleven months. Such dosing is currently believed to be particularly suited for canines, but is believed to also be applicable in other mammals, namely, felines, equines and human beings. As to the dosage of such vaccine, it is contemplated that a cellular dosage of 3×10⁶ cells will provide sufficient concentrations of the aforementioned antigens which will elicit the desired immune response. It should be understood, however, that the fine tuning of the administration of such vaccine can be determined and practiced by one of ordinary skill in the art by following the effectiveness of the vaccine in various clinical settings and in the context of the features of the tumor in the subject being treated.

### DETAILED DESCRIPTION OF THE INVENTION

The following detailed description is provided for purposes of illustrating and describing presently preferred embodiments of the invention and are not intended to limit the scope of the invention in any way. It will be recognized that further embodiments of the invention may be used.

A new vaccine is disclosed for use in the treatment of certain malignant solid tumors found in mammals, namely, melanoma, carcinoma, mast cell tumors, and sarcoma. Although presently believed to be particularly effective in treating canines, it is currently believed that such vaccination will also be particularly well suited for use with felines, equines and human beings. At the outset, it should be understood that the term “vaccine” as used herein is meant to refer to a compound or composition, as appropriate, capable of conferring a degree of specific immunity when administered to a mammalian host. To that end, the vaccines of the present are capable of stimulating a specific immunological response (such as a cellular or humoral anti-tumor antigen or anti-cancer cell response) mediated at least in part by the immune system of the host individual. The immunological response may comprise antibody production, proliferation of immuno-reactive cells, or any combination thereof, and is preferably directed towards at least one or more tumor-associated antigens present on a tumor for which treatment is directed.

The vaccine preferably consists of at least one allogenic cell line which is known to contain at least one tumor-associated antigen, and more particularly, at least one ganglioside demonstrated to be immunogenic (i.e., capable of generating an immune response against such antigen) in subjects afflicted with either melanoma, sarcoma, or carcinoma. Such tumor-associated antigen will be present in effective amounts sufficient to elicit an immune response thereto.

Preferably, the cell line will contain at least two gangliosides which are preferably selected from a group consisting of GD-2, GD-3, GM-2, GM-3, GD-1b, and GT-1b. At present, it is believed that even so much as one ganglioside, namely GM-2, may be sufficient to elicit the desired immune response when coupled with other adjuvants, discussed more fully below. Accordingly, ganglioside GM-2 should be considered most preferred among the aforementioned group of gangliosides. Following GM-2, GD-2 is believed to be the ganglioside which helps elicit the most tumor-specific immune response. GD-3, based upon present understanding, would appear to be the third most desirable tumor-associated antigen to be exhibited by the cell line. To further enhance the immunogenic response generated by the cellular component of the vaccine of the present invention, such cellular component may further preferably include leukocyte antigens (LA) that are common with the subject to which the vaccine is administered. In this regard, such antigens will not only be species specific, but correspond to the particular antigens of the subject, which will be readily appreciated by one skilled in the art.

One suitable canine cell line for use in canine vaccination applications can be procured from Animal Science Laboratories of Palm Desert, Calif. Additionally, upon allowance and issuance of this application as a United States patent, the identity of a cell line, its description and ATCC accession number will be identified and all restriction on the availability of such deposit will be irrevocably removed. Furthermore, access to the designated deposit will be available during pendancy of the above-named application to one determined by the Commissioner to be entitled thereto, under 37 C.F.R. § 1.14 and 35 USC § 1.22. Moreover, the designated deposit will be maintained for a period of thirty (30) years from the date of deposit, or from five (5) years after the last request for the deposit; or for the enforceable life of the patent issuing on this application, whichever is longer.

At present, it is believed that a dosage of such cells would comprise 3×10⁶ cells. In this regard, the cellular vaccines of the present invention are typically assembled by preparing each cell population or equivalent thereof in an appropriate fashion which are within the skill of the art. Such cells are further preferably irradiated to 150 Gy to thus render the same incapable of proliferation, as per conventional practices. The cells may be suspended in an excipient containing 10% dimethylsulfoxide and thereafter may be cryo-preserved in liquid nitrogen, again according to standard practice.

In addition to the cell line, at least one adjuvants must necessarily be included as part of the practice of the present invention. Specifically, the present invention contemplates co-administration of at least one cytokine and heat shock protein. With respect to the former, the cytokine is preferably selected from the group consisting of GM-CSF, IL-2, IL-4, and IL-12. As is well known in the art, such ctyokines augment the immune response to tumor-associated antigens, such as through T-cell activation and proliferation (IL-2) or dendritic cell attraction and maturation (GM-CSF). Of such cytokines, GM-CSF is believed to be most preferred, followed by IL-2. It should be understood, however, that additional cytokines, such as tumor necrosis factors and/or interferons could additionally be incorporated into the practice of the present invention to the extent such agents produce biological activities which enhance the immune response, whether by enhancing proliferation of lymphocytes, enhance the ability to uptake antigen-presenting cells, and/or enhances the ability of tumor cells to display tumor-associated antigens, other desirable immunological effects.

With respect to the heat shock protein adjuvant, the same preferably includes at least one protein selected from the group consisting of HSP-60, HSP-70 and HSP-90, although other heat shock proteins are contemplated. As is currently being recognized, such heat shock proteins appear to play important roles in immunity and are major targets of immune responses to a wide variety of pathogens. Indeed, it is believed that at least one heat shock protein is vital to the functioning of the vaccine of the present invention. To that end, HSP-90 is currently believed to be preferred in the practice of the present invention.

Additionally preferred, but not essential to the desired immunogenicity generated by the vaccine of the present invention, is the co-administration of a bacterial agent to activate the immune system in a non-specific fashion which serve to augment the immune response to tumor-associated antigens. Among the bacterial agents considered ideal for the practice of the present invention include Bacillis Calmette-Gruen (BCG). Although currently not tested, it is believed that other well-known bacterial agents, such as Corynabacterium parvum (C. parvum) and staphylococcus aureus may also be utilized as an adjuvant to facilitate the immune response.

It is further contemplated that as an additional adjuvant, a melanoma associated antigen may be included for the vaccination of melanoma. Specifically, it is contemplated that one of at least three specific antigens, namely, GP-100, MAGE-1, and/or MART-1 may be included to help facilitate and elicit the desired immune response. It is further contemplated that additional melanoma associated antigens, such as BAGE, GAGE, and/or tyrosinase may further be utilized. Such antigenic peptides are known in the art and currently in use in clinical trials in patients with metastatic melanoma.

In any embodiment, the cellular vaccines of the present invention will typically be assembled by preparing each cell preparation or equivalent thereof in an appropriate fashion and combining the components for administration to a subject. In this regard, each of the components will be present in an effective amount sufficient to affect the beneficial or desired clinical result, particularly the generation of an immune response, or noticable improvement in clinical condition. In terms of clinical response, for subjects afflicted with melanoma, carcinoma, mast cell tumors or sarcoma, an effective amount is an amount sufficient to palliate, ameliorate, stabilize, reverse or slow progression of the disease, or otherwise reduce pathological consequences of the disease.

To that end, an effective amount of the vaccine in the present invention may comprise either a single or divided dose. Presently, it is contemplated that the vaccine of the present invention should be administered on a weekly basis for five weeks, and thereafter on a monthly basis from ten to eleven months. It is further believed that this dosage is applicable to all of the aforementioned cancers discussed herein, namely melanoma, mast cell tumors, carcinoma and sarcoma. Likewise, it is believed that such vaccine regimen would be applicable to other species, which at present would comprise feline, equine and possible human beings.

As discussed above, the vaccine of the present invention is preferably administered as a cellular vaccine, with the allogenic cell component thereof maintained structurally intact. It is contemplated, however, that such component may be administered as a cellular lysate or even administered as a vaccine with the aforementioned antigens and adjuvants adsorbed upon a suitable pharmaceutically-acceptable carrier. Along these lines, it should be recognized that the practice of the present invention employs and can employ conventional techniques of molecular biology, microbiology, cell biology, biochemistry and immunology which are within the skill of the art.

As a further guide to a practitioner of ordinary skill in the art, the following case studies are presented below.

### CASE STUDIES

As examples of the efficacy of the vaccine treatment, two dogs presenting with mast cell sarcoma underwent treatment with the vaccine, with positive results.

In May of 2001, Sasha Lee, a 4 year old female golden retriever, presented with grade II mast cell sarcoma, consisting of a primary tumor below the left eye. The tumor was excised by her veterinarian and no additional treatment was requested by the owner. In June of 2001, Sasha presented with two open and bleeding lesions on her back, indicating progression of the disease to stage III. The vaccine, including cells expressing gangliosides GD-2, GM-2 and GD-3 with GM-CSF, HSP-90 and 25,000 cfu of BCG was administered to the left mandibular lymph node. Seven days later, Sasha presented with drying and a 50% reduction in the two lesions, but eight new tumors appeared on her back. Again, the vaccine with 25,000 cfu of BCG was administered to the left mandilla lymph node. Seven days later, the two initial lesions were dried and healed and the eight subsequent tumors were gone. The vaccine was administered to the left mandilla lymph node, without BCG. Three more treatments of the vaccine (without BCG) were administered at seven days, eight days and one month after the disappearance of all lesions. A follow-up examination approximately six months after the first discovery of a lesion has shown no evidence of the initial lesions nor any appearance of new lesions.

In July of 2001, Annie Jenkins, a seven year old female canine of mixed breed, presented with a primary lesion diagnosed as grade II mast cell carcinoma. Two weeks after the initial discovery, a vaccine using the same cell line, cytokine and heat shock protein discussed above, along with 25,000 cfu of BCG, was administered to the left mandibular lymph node. Another similar dose was given seven days later, and no evidence of the tumor was present. Two more doses of the vaccine, without BCG, were given at seven day intervals and as of late October, 2001, there was no evidence of the tumor.

Although the foregoing invention has been described in detail by way of illustration and example, it will be apparent to those skilled in the art that certain changes and modifications may be practiced. Therefore, the description examples set forth herein should not be construed as limiting the scope of the invention, but rather as defined by the appended claims.

What is claimed is:

1. A composition for inducing an immune response in a mammal to solid tumor associated antigens, the composition comprising:
(a) at least one allogenic line of cancer cells providing at least one tumor-associated ganglioside selected from the group consisting of GD-2, GD-3, GM-2, GM-3, GD-1b, and GT-1b;
(b) at least one cytokine selected from the group consisting of GM-CSF, IL-2, IL-4, IL-12 and TNF∝; and
(c) at least one heat shock protein selected from the group consisting of HSP-60, HSP-70 and HSP-90.

2. The composition of claim 1 wherein said allogenic cell line provides at least two gangliosides selected from said group of gangliosides.

3. The composition in claim 1 wherein said ganglioside comprises GM-2.

4. The composition in claim 1 wherein said ganglioside comprises GD-2.

5. The composition in claim 2 wherein said gangliosides comprise GM-2 and GD-2.

6. The composition in claim 1 wherein said allogenic cell line provides at least three gangliosides selected from said group of gangliosides.

7. The composition in claim 6 wherein said gangliosides comprise GM-2, GD-2 and GD-3.

8. The composition in claim 1 wherein said cytokine comprises GM-CSF.

9. The composition of claim 1 wherein said cytokine comprises IL-2.

10. The composition of claim 1 wherein said composition comprises at least two cytokines selected from said group of cytokines.

11. The composition of claim 10 wherein said cytokines comprise GM-2 and IL-2.

12. The composition of claim 1 wherein said heat shock protein comprises HSP-90.

13. The composition of claim 1 further comprising a bacterial adjuvant.

14. The composition of claim 13 wherein said bacterial adjuvant comprises BCG.

15. The composition of claim 1 wherein said allogenic cell line further provides leukocyte antigens in common with the mammal to which the composition is administered.

16. The composition of claim 1 wherein said allogenic cell line is rendered incapable of proliferation by radiation.

17. The composition of claim 1 wherein said mammal comprises a canine and said allogenic cell line comprises a canine cancer cell line.

18. A method of inducing a systemic immune response in a mammal to solid tumor associated antigens, the method comprising the steps:
(a) providing cells of at least one allogenic cell line providing at least one tumor-associated ganglioside, selected from the group consisting of GD-2, GD-3, GM-2, GM-3, GD-1b, and GT-1b;
(b) providing at least one cytokine selected from the group consisting of GM-CSF, IL-2, IL-4, IL-12 and TNF∝;
(c) providing at least one heat shock protein selected from the group consisting of HSP-60, HSP-70 and HSP-90; and
(d) concurrently administering an effective number of cells in step (a), an effective amount of said cytokine in step (b), and an effective amount of said heat shock protein in step (c) to said mammal, said administration thereby inducing a systemic immune response specific to said antigens.

19. The method of claim 18 wherein said allogenic cell line provides at least two gangliosides selected from said group of gangliosides.

20. The method in claim 18 wherein in step (a) said ganglioside comprises GM-2.

21. The method in claim 18 wherein in step (a) said ganglioside comprises GD-2.

22. The method in claim 19 wherein in step (a) said gangliosides comprise GM-2 and GD-2.

23. The method in claim 18 wherein in step (a) said allogenic cell line provides at least three gangliosides selected from said group of gangliosides.

24. The method in claim 23 wherein in step (a) said gangliosides comprise GM-2, GD-2 and GD-3.

25. The method in claim 18 wherein in step (b) said cytokine comprises GM-CSF.

26. The method of claim 18 wherein in step (b) said cytokine comprises IL-2.

27. The method of claim 18 wherein in step (b) said composition comprises at least two cytokines selected from said group of cytokines.

28. The method of claim 27 wherein in step (b) said cytokines comprise GM-CFS and IL-2.

29. The method of claim 18 wherein in step (c) said heat shock protein comprises HSP-90.

30. The method of claim 18 further comprising step (e): providing a bacterial adjuvant and co-administering said adjuvant in step (d).

31. The method of claim 31 wherein said bacterial adjuvant comprises BCG.

32. The method of claim 18 wherein in step (a) said allogenic cell line further provides leukocyte antigens in common with the mammal to which the composition is administered.

33. The method of claim 18 wherein in step (a) said allogenic cell line is rendered incapable of proliferation by radiation.

34. The method of claim 18 wherein said mammal comprises a canine and in step (a) said allogenic cell line comprises a canine cancer cell line.

35. The method of claim 18 wherein in step (a), said allogenic cell line comprises approximately 3×10⁶ allogenic cells.

36. The composition of claim 1 further comprising a melanoma-associated antigen.

37. The composition of claim 36 wherein said melanoma-associated antigen is selected from the group consisting of MAGE-1, MART-1 and GP-100.

38. The method of claim 18 further comprising step (e): providing a melanoma-associated antigen and co-administering said antigen in step (d).

39. The method of claim 38 wherein said melanoma-associated antigen is selected from the group consisting of MAGE-1, MART-1 and GP-100.
